(19) United States
(12) Patent Application Publication
(10) Pub. No.: US 2008/0275227 A1
(43) Pub. Date: Nov. 6, 2008

(54) Process for Modifying an Organic Natural Substance by Exchange Reactions

(76) Inventor: Emil Joas, Vocklabruck (AT)

Correspondence Address: COLLARD & ROE, P.C., 1077 NORTHERN BOULEVARD, ROSLYN, NY, 11576, US

(21) Appl. No.: 11/884,692

(22) PCT Filed: Feb. 21, 2006
(86) PCT No.: PCT/AT2006/000065

§ 371 Date: Apr. 11, 2008

(30) Foreign Application Priority Data
Feb. 21, 2005 (AT) A 283/2005

(51) Int. Cl. C07H 1/00; B01J 19/00

(52) U.S. Cl. 536/18.5; 422/187

(57) ABSTRACT

A process for modifying an organic natural substance by exchange reactions is characterised in that the natural substance to be modified is heated in the liquid phase as an aerosol in a gas stream, with the exclusion of air, along a conveyance path and, after reaching a temperature dependent on the bond energy of the molecules and/or molecular groups to be exchanged, is removed from the conveyance path to supply to the natural substance to be modified the materials required for the exchange reaction under the respectively required reaction conditions, and in that the reaction product is then supplied to the conveyance path in the circuit and heated to an elevated reaction temperature for carrying out the exchange reactions before the reaction product is withdrawn from the conveyance path and condensed to obtain the modified natural substance.

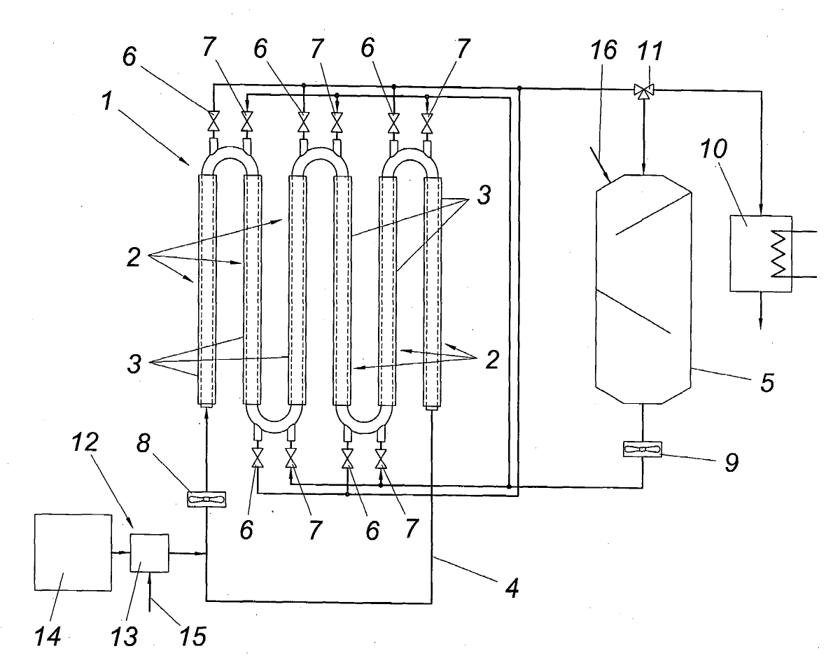

### 1. FIELD OF THE INVENTION

The invention relates to a process for modifying an organic natural substance by exchange reactions.

### 2. DESCRIPTION OF THE PRIOR ART

Natural substances obtained from plants frequently have to be modified in order to be able to be ingested by the human organism without impairing health. Stevioside is thus, for example, a sweetener which has a very high sweetening effect and can be obtained from an aqueous extract of the leaves of the half-shrub Stevia rebaudiana by precipitation with alcohol and freezing-out. Stepvioside is a glucoside consisting of glucose, sophorose and steviol. Nevertheless, the steviol contained in stevioside is, as a mutagenic phytohormone, a problematic substance which has to be inactivated or removed for stevioside to be used as a sweetener. Conventional processes for the separation or exchanging of molecules and/or molecular groups of a natural substance require, in addition to a comparatively high use of energy, corresponding chemical means which entail complex secondary cleaning processes. Also, the molecular structure of the separated or exchanged substances is usually altered in such a way that the substances can be disposed of only as waste.

### SUMMARY OF THE INVENTION

The object of the invention is therefore to configure a process for modifying an organic natural substance by exchange reactions so as to allow molecules or molecular groups to be separated in a simple manner, using comparatively little energy, from the natural substances to be modified and to be replaced by suitable substances without having to allow for complex secondary cleaning processes.

The invention achieves the object set in that the natural substance to be modified is heated in the liquid phase as an aerosol in a gas stream, with the exclusion of air, along a conveyance path and, after reaching a temperature dependent on the bond energy of the molecules and/or molecular groups to be exchanged, is removed from the conveyance path to supply to the natural substance to be modified the materials required for the exchange reaction under the respectively required reaction conditions, and in that the reaction product is then supplied to the conveyance path in the circuit and heated to an elevated reaction temperature for carrying out the exchange reactions before the reaction product is withdrawn from the conveyance path and condensed to obtain the modified natural substance.

The invention is based on the finding that exchange reactions can be advantageously initiated if the bonds of the molecules or molecular groups to be exchanged are broken to such an extent as substantially to produce a state of equilibrium between decomposition and renewal of the molecular bonds. If, therefore, the exchange substances are offered to the natural substances in this excited state and the molecular bonds of the substances to be exchanged are then broken up more extensively, the supplied exchange substances can be linked quickly and effectively to the natural substances to be modified. In order to be able to utilise this circumstance, the natural substances to be modified are heated in the liquid phase as aerosols in a gas stream along a conveyance path until there is reached a temperature which is dependent on the respective bond energy of the molecules and/or molecular groups to be exchanged and brings about the desired partial cancellation of the bond strength. To supply the substances required for the exchange reaction, the gas stream comprising the aerosols is withdrawn from the conveyance path so as to ensure advantageous reaction parameters for the subsequent exchange reaction.

To shift the reaction equilibrium with respect to the desired exchange reaction, there can be taken outside the conveyance path various measures, such as the addition of enzymes or the use of ionising radiation, which assist the subsequent exchange reactions at an elevated reaction temperature. The fact that the natural substances are in aerosol form not only facilitates the transition of heat but is also a basic requirement for thorough mixing of the natural sub-stances with the substances involved in the exchange reaction, especially if these substances are also used as aerosols. After a corresponding reaction time, the reaction product can be withdrawn again for the conveyance path and condensed to obtain the modified natural substance. The modified natural sub-stance is in this case separated from the exchanged substances, the exchange substances which are still present, any additives and the remaining content of the non-modified natural substance, wherein the non-modified content of the natural substance and the exchange substances which are still present can be resubjected to the exchange reaction described, whereas the remaining sub-stances separated from the modified natural substance are supplied to a further-processing means.

As the bond strength of differing molecules or molecular groups varies, the individual partial reactions are, in the case of multistage exchange reactions, carried out in succession, in accordance with their differing reaction temperatures, while passing repeatedly over the conveyance path once the respectively required exchange substances have been supplied at a temperature below the respective reaction temperature. Under the above-described process conditions, a multistage exchange reaction is thus possible under advantageous production conditions. The conveyor gas stream for the aerosols to be treated comprises a gas which at least does not impair the desired exchange reaction and the composition of which can be altered to assist the respective exchange reaction in conjunction with the supply of the substances, required for the exchange reaction, to assist the exchange reaction. For many applications, the use of carbon dioxide as the conveyor gas has proven effective, wherein carbon dioxide can also advantageously be used to atomise a preferably aqueous solution of the natural substance to be modified.

To modify an organic natural substance by exchange reactions using the process according to the invention, the starting point may be a device having a heatable conveyance path for the natural substance fed to a conveyor gas stream via a supply means, comprising a reactor linked in a circuit to the conveyance path and comprising a condenser connected to the conveyance path. If the supply means is provided with a means for atomising the liquid natural substance, the conveyance path having a plurality of portions which can be heated individually and can each selectively be connected to the reactor or the condenser, all of the requirements are met for a sequence of processes which can be adapted to the respective process conditions to modify a natural sub-stance by an exchange reaction. The natural substance, which is provided in the liquid phase, can be introduced as an aerosol into the conveyor gas stream via the atomisation means, wherein the heating of the conveyor gas stream and the aerosols to the respective treatment temperature in the individual portions, each of which can be heated, of the conveyance path can be carried out in a sensitive manner. The connection, both on the outlet and on the inlet side, of the individual portions of the conveyance path to the reactor allows differing process implementation, adapted to each application, which allows for each process step the respective required process temperature to be adhered to, at a corresponding heating characteristic, because the gas stream is able to flow through the individual portions of the conveyance path even when they are arranged groupwise in rows. The condenser requires merely a connection to the outlet side of the individual portions of the conveyance path.

### BRIEF DESCRIPTION OF THE DRAWING

The process according to the invention will be described in greater detail with reference to the drawing showing a device according to the invention for modifying an organic petrol substance by exchange reactions.

### DESCRIPTION OF THE PREFERRED EMBODIMENT

According to the illustrated embodiment, the device for modifying an organic natural substance has a conveyance path 1 for the natural substance to be treated which is conveyed through the pipeline forming the conveyance path 1 using a gas stream as the means of conveyance. The conveyance path 1 is divided into a plurality of portions 2 which are each individually heatable. For this purpose, there is associated with each portion 2 an electric heating means 3 which heats the jacket of the pipeline forming the conveyance path 1, so the jacket acts as a heating surface for heating by thermal radiation the gas stream loaded with the natural substance to be treated. The individual portions 2 are connected—apart from the inlet and outlet-side portions which are joined together by a circuit line 4—to a reactor 5 on the outlet and inlet sides, on the one hand via control members 6 for removing a flow of products from the respective portion 2 and on the other hand via control members 7 for introducing a flow of products which can thus be respectively guided in a circuit via the reactor 5. The flow of products are conveyed along the conveyance path 1 via the circuit line 4 using a fan 8. A fan 9 is provided for conveying the flow of products through the reactor 5. The individual portions 2 are connected via the control members 6, which, like the control members 7, are preferably in the form of control valves, not only to the reactor 5 but also to a condenser 10, wherein the flow of products withdrawn from the respective portions 2 can selectively be supplied to the reactor 5 or the condenser 10 via a switching means 11.

The natural substance to be treated is fed to the conveyor gas stream via a supply means 12 which consists of a means 13 for atomising the natural sub-stance which is in the liquid phase, is removed from a storage container 14 and is preferably atomised using the conveyor gas fed to the atomisation means 13 via a pressure line 15, so the natural substance to be treated is in the form of an aerosol. The aerosols fed to the conveyor gas stream are heated along the conveyance path, ensuring, using the individually heatable portions 2, sensitive heating to the respectively provided temperature. In order to be able advantageously to carry out exchange reactions, the flow of products is initially heated to a temperature at which the bonds of the molecules or molecular groups to be exchanged are broken up to such an extent as substantially to produce a state of equilibrium between breaking-up and restoration of the molecular bonds; this can be monitored using infrared spectrometry based on the concentration of the separated molecules or molecular groups. In this excited state, the flow of products is supplied to the reactor 5 into which the substances required for the exchange reaction are supplied, as is indicated by the inlet 16. As there are defined in the reactor 5 advantageous conditions for the subsequent exchange reaction which is carried out at an elevated reaction temperature, the reaction equilibrium is shifted under the terms of the desired exchange reaction. For carrying out the exchange reaction in the circuit, the reaction product is supplied to the conveyance path 1, into the portion 2 which is respectively suitable for heating the reaction product. Once the exchange reaction has been carried out to a sufficient extent, monitored by infrared spectrometry, the reaction product is withdrawn again from the conveyance path 1 in order either to initiate a further reaction stage via the reactor 5 or to feed the treatment product to the condenser 10. From the condensate obtained, the natural substance modified by the exchange substances can then be separated from the remaining sub-stances using known processes.

### Embodiment

A concentrated aqueous stevioside solution was atomised using carbon dioxide in a conveyor gas stream consisting of carbon dioxide, thus allowing the stepvioside to be treated to be heated in aerosol form along the conveyance path 1, continuously to 98° C., a pH between 5.8 and 6.0 being set, optionally by adding spirit vinegar. The flow of products heated to this treatment temperature was then guided into the reactor 5 which had been heated to the same temperature. In the reactor 5, there was added to the flow of products an aqueous mannitol solution which was atomised at the given treatment temperature using carbon dioxide. Mannitol, which is a sweetener harmless to humans and is intended to replace the steviol contained in stevioside, was supplied in an excessive amount with regard to the exchange reaction, wherein the pH could if necessary be corrected by adding acetic acid. In order to obtain conditions advantageous for the exchange reaction, the reactor may contain five substrate-specific catalysts which, for the desired modification of stevioside, can consist of aluminium or a copper alloy. After the addition via the inflow 16 of the substances required for the exchange reaction, the reaction product was fed again to the conveyance path 1, the portions 2 of which had been heated to temperature values of between 100 and 125° C. in order to heat the reaction product to the desired reaction temperature. The extent of the exchange reaction could be monitored using infrared spectrometry, so at a degree of conversion of at least 60%, which was achieved after 20 to 30 min, the reaction product could be withdrawn in order, after neutralisation with lime, to carry out condensation in the condenser 10. The further separation was carried out by conventional recrystallisation processes by which the modified stevioside comprising mannitol instead of steviol, the non-converted stevioside, the released steviol and the excess mannitol is obtained. The non-converted stevioside comprising the mannitol could again be subjected, in a subsequent batch, to the described exchange reaction. The steviol can be used, inter alia, as a partial component in cosmetics.

1. Process for modifying an organic natural substance by exchange reactions, wherein the natural substance to be modified is heated in the liquid phase as an aerosol in a gas stream, with the exclusion of air, along a conveyance path and, after reaching a temperature dependent on the bond energy of the molecules and/or molecular groups to be exchanged, is removed from the conveyance path to supply to the natural substance to be modified the materials required for the exchange reaction under the respectively required reaction conditions, and in that the reaction product is then supplied to the conveyance path in the circuit and heated to an elevated reaction temperature for carrying out the exchange reactions before the reaction product is withdrawn from the conveyance path and condensed to obtain the modified natural substance.

2. Process according to claim 1, wherein, in the case of multi-stage exchange reactions, the individual partial reactions are carried out in succession, in accordance with their differing reaction temperatures, while passing repeatedly over the conveyance path once the respectively required exchange substances have been supplied at a temperature below the respective reaction temperature outside the conveyance path.

3. Process according to claim 1, wherein a solution of the natural substance to be modified is atomized using carbon dioxide to form an aerosol and conveyed along the conveyance path using the carbon dioxide as the conveyor gas.

4. Device for carrying out the process for modifying an organic natural substance by exchange reactions using a heatable conveyance path for the natural substance fed to a conveyor gas stream via a supply means, comprising a reactor linked in a circuit to the conveyance path and comprising a condenser connected to the conveyance path, wherein the supply means (12) contains a means (13) for atomizing the liquid natural substance and in that the conveyance path (1) has a plurality of portions (3) which can be heated individually and can each selectively be connected to the reactor (5) or the condenser (10).
